(19) United States
(12) Patent Application Publication
(10) Pub. No.: US 2026/0315185 A1
(43) Pub. Date: Oct. 15, 2026

(54) PIEZOELECTRIC DENTAL SURGICAL TOOL AND METHOD OF USE

(71) Applicant: The University of Melbourne, Victoria (AU)

(72) Inventors: Scott Gohery, Victoria (AU); Efthimios Gazelakis, Victoria (AU); Mohsin Nazir, Victoria (AU); Roy Judge, Victoria (AU)

(73) Assignee: The University of Melbourne, Victoria (AU)

(21) Appl. No.: 19/119,071

(22) PCT Filed: Oct. 6, 2023
(86) PCT No.: PCT/AU2023/050981

§ 371 Date: Apr. 7, 2025

(30) Foreign Application Priority Data
Oct. 7, 2022 (AU) 2022902930

(51) Int. Cl. A61C 1/07; A61B 17/16; A61B 17/32

(52) U.S. Cl. CPC A61C 1/07; A61B 17/1673; A61B 17/320068; A61B 2017/320082

(57) ABSTRACT

A piezoelectric surgical tool tip 10, comprising a tool tip body 20 having a proximal end 22 configured for connection with a tool handle 32 of a powered piezoelectric surgery unit 30 and a distal end 24 configured for cutting into an alveolar bone 34 of a patient using micro-vibrations generated by the surgery unit 30 and tool handle 32, the distal end 24 comprising an outwardly extending arm 26 and a base 40 on which extends a plurality of cutting projections 42 arranged in the form of an array 44, wherein the base 40 is mounted substantially perpendicular to the arm 26 and the array 44 is substantially rectangular in shape with a maximum width of about 6 mm and a maximum length of about 5 mm.

### FIELD OF THE INVENTION

This invention relates to a piezoelectric dental surgical tool system and its method of use, and in particular surgical tool tips and their method of use for creating horizontally oriented rectangular dental osteotomy sites in alveolar bone.

### BACKGROUND

Implanting a dental implant can involve the removal of bone tissues from the alveolar bone, which contains the tooth sockets, using specialised piezoelectric dental tools and tool tips. Typically, osteotomy sites in the form of vertical sockets are excavated from the bone tissues by dental practitioners to house the artificial tooth implants. The conventional tool tips used to create these vertical sockets are designed to cut deep into bone tissue but are not suitable for cutting effectively across a wider surface area. It has been found that conventional short implants installed this way into vertical osteotomy sites require a minimum height clearance in the bone to secure the implant, and implants installed without a sufficient bone height are less able to withstand loads applied over time and have a tendency to fail in the long term.

An improved horizontally-oriented implant design was conceived and published in PCT application no. PCT/AU2016/050339, the entire contents of which are hereby incorporated into this specification by reference. The improved implant design overcomes a number of shortcomings of conventional short vertically-oriented implants, including the requirement for a large bone height clearance for the osteotomy site, and addresses the implant design parameters of maximal utilisation of the remaining bone volume, minimisation of risk to the Inferior Alveolar Nerve (IAN) or other vital structures in or near the oral cavity, and maximisation of force distribution of the implant. However, conventional dental surgical tools and tool tips are not adequate for the preparation of a suitable osteotomy site to accommodate the improved horizontally-oriented implant design.

Therefore, there exists a need for a solution that provides a safe, effective and reliable way to create dental osteotomy sites in the alveolar bone for the newly improved horizontally-oriented implant designs. The solution should ideally be compatible with existing piezoelectric dental surgical systems, easy to learn and familiar to dental practitioners.

The applicant has determined that it would be advantageous to provide improved surgical tool tips and new methods of using the tool tips which, in the preferred embodiments, seek to at least in part alleviate the above-identified problems or to offer the public with a useful choice.

### SUMMARY OF THE INVENTION

According to an aspect of the present invention, there is provided a piezoelectric surgical tool tip 10, comprising a tool tip body 20 having a proximal end 22 configured for connection with a tool handle 32 of a powered piezoelectric surgery unit 30 and a distal end 24 configured for cutting into an alveolar bone 34 of a patient using micro-vibrations generated by the surgery unit 30 and tool handle 32, the distal end 24 comprising an outwardly extending arm 26 and a base 40 on which extends a plurality of cutting projections 42 arranged in the form of an array 44, wherein the base 40 is mounted substantially perpendicular to the arm 26 and the array 44 is substantially rectangular in shape with a maximum width of about 6 mm and a maximum length of about 5 mm.

In some embodiments, the cutting projections of the array are arranged linearly in series of rows and columns, defined relative to the orientation of the tool tip when the tool tip body is in an upright position.

In some embodiments, the array of cutting projections is configured in the form of a tall rectangular shape in which the number of rows is greater than the number of columns, and wherein the width of the array is about 4 mm and the length of the array is about 5 mm.

In some embodiments, wherein the number of columns of cutting projections is five and the number of rows of cutting projections is seven.

In some embodiments, the array of cutting projections is configured in the form of a wide rectangular shape in which the number of columns is greater than the number of rows, and wherein the width of the array is about 6 mm and the length of the array is about 3 mm.

In some embodiments, the number of columns of cutting projections is six and the number of rows of cutting projections is five.

In some embodiments, each of the plurality of cutting projections is generally shaped in the form of a triangular prism and oriented such that an upper vertex of the prism defines a cutting edge.

In some embodiments, a row or column of like triangular prism cutting projections is formed such that their triangular bases are integrally connected.

In some embodiments, a gap of about 0.75 mm is provided between the cutting edges of adjacent like triangular prisms in the array.

In some embodiments, each cutting projection is configured with a height of about 1.2 mm.

In some embodiments, the arm is in the form of a flattened beam, and wherein the arm is provided with one or more marking(s) located along the beam to indicate a relative distance from the marking to the array of cutting projections.

In some embodiments, the or each marking is a cavity that extends through the arm.

In some embodiments, the arm is configured with a thickness of about 0.75 mm.

In some embodiments, the tool tip body further comprises an internal passageway for receiving water flow at the proximal end and guiding said water flow generally towards the distal end.

In some embodiments, the array of cutting projections and the base are integrally formed.

In some embodiments, the tool tip body is treated with a titanium nitride coating.

In some embodiments, the tool tip body is configured to be detachably connectable with the tool handle of the powered piezoelectric surgery unit.

According to another aspect of the present invention, there is provided a dental surgical system, comprising a piezoelectric surgery unit configured for generating predetermined ultrasound frequencies; a tool handle connectable to the piezoelectric surgery unit to generate micro-vibrations at the predetermined frequencies; and a piezoelectric surgical tool tip as previously described configured for connection with the tool handle for cutting into an alveolar bone of a patient using the generated micro-vibrations.

According to yet a further aspect of the present invention, there is provided a method of creating a generally horizontally rectangular dental osteotomy site in an alveolar bone, the method comprising the steps of providing a first piezoelectric surgical tool tip as previously described in which the array of cutting projections is configured in the form of a tall rectangular shape having a width of about 4 mm and a length of about 5 mm; providing a second piezoelectric surgical tool tip as previously described in which the array of cutting projections is configured in the form of a wide rectangular shape having a width of about 6 mm and a length of about 3 mm; applying piezoelectric vibrations to cut the alveolar bone using the first piezoelectric surgical tool tip in a first direction, which is substantially along a mesial-distal direction of the alveolar bone; applying piezoelectric vibrations to cut the alveolar bone using the second piezoelectric surgical tool tip in a second direction, which is substantially perpendicular to the first direction; and alternating applications of piezoelectric vibrations using the first and second piezoelectric surgical tool tips to the alveolar bone until a generally horizontally rectangular osteotomy site with a desired depth is achieved.

In some embodiments, the method further comprises the step of applying piezoelectric vibrations to the horizontally rectangular osteotomy site using a piezoelectric surgical tool tip with a chisel tip end to smooth out any irregular surfaces within the site.

Further aspects of the present invention and further embodiments of the aspects described in the preceding paragraphs will become apparent from the following description.

While components of the piezoelectric dental surgical tool will be described below for use in combination with each other in the preferred embodiments of the present invention, it is to be understood by a skilled person that some aspects of the present invention are equally suitable to be used interchangeably between one or more embodiments of the present invention and/or suitable for use as standalone inventions that can be individually incorporated into other piezoelectric surgical tools not described herein.

The word “about” or “approximately” when used in relation to a stated reference point for a quality, level, value, number, frequency, percentage, dimension, location, size, amount, weight or length may be understood to indicate that the reference point is capable of variation, and that the term may encompass proximal qualities on either side of the reference point.

As used herein, the words “substantially” and “generally” may be used merely to indicate an intention that the term it qualifies should not be read too literally and that the word could mean “sufficiently”, “mostly” or “near enough” for the patentee's purposes.

### DESCRIPTION OF THE DRAWINGS

The invention will now be described, by way of non-limiting example only, with reference to the accompanying drawings, in which:

FIG. 1 is a schematic view of a piezoelectric dental surgery unit including a tool handle and a tool tip connected to the handle;

FIG. 2 is a schematic sectional view of an alveolar bone and the potential site of a generally horizontal rectangular osteotomy site;

FIG. 3 is a perspective view of a piezoelectric tool tip in accordance with a preferred embodiment of the present invention;

FIG. 4 is a close up perspective view of the tool tip showing an array of cutting projections;

FIG. 5 is another close up perspective view of the array of cutting projections of the tool tip;

FIG. 6 is a side perspective view of the cutting projections of the tool tip;

FIG. 7 is a partially exploded view of the tool tip showing the array of cutting projections;

FIG. 8 is a another side view of the tool tip;

FIG. 9 is a perspective view of the tool tip from below showing the tool tip base portion;

FIG. 10 is a sectional view of the tool tip of FIG. 3;

FIG. 11 is a perspective view of a piezoelectric tool tip in accordance with another preferred embodiment of the present invention;

FIG. 12 is a close up perspective view of the tool tip of FIG. 11 showing an array of cutting projections;

FIG. 13 is a side perspective view of the tool tip of FIG. 11;

FIG. 14 is a perspective view of a piezoelectric tool tip having a chisel end according to another embodiment of the present invention;

FIG. 15 is a close up perspective view of the tool tip of FIG. 14;

FIG. 16 is a further perspective view from above of the tool tip of FIG. 14;

FIG. 17 is a perspective view of a piezoelectric tool tip having a chisel end in another orientation according to yet a further embodiment of the present invention;

FIG. 18 is a close up perspective view of the tool tip of FIG. 17; and

FIG. 19 is a further perspective view from above of the tool tip of FIG. 17.

### DETAILED DESCRIPTION

The present invention relates to new piezoelectric dental surgical tool tips for use with a powered piezoelectric surgery system and a method of creating a generally horizontal rectangular dental osteotomy site, having an depth of about 5.5 mm, in an alveolar bone of a patient using the tool tips as described.

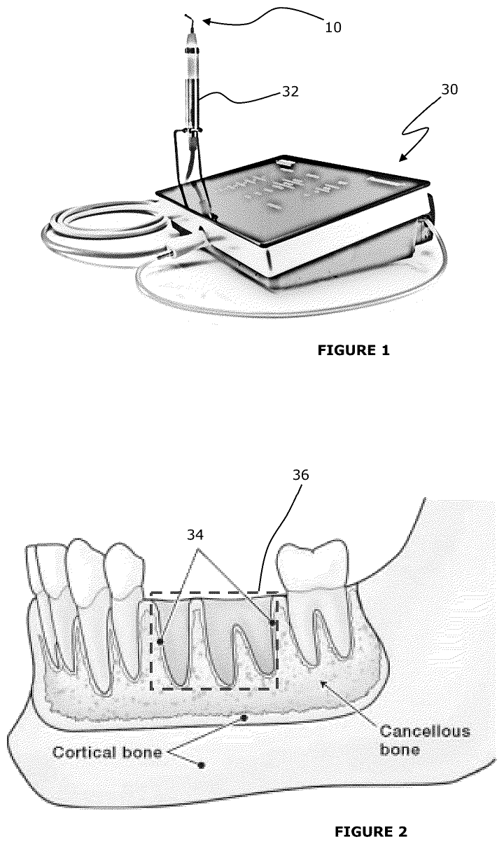

FIG. 1 shows a piezoelectric dental surgery unit 30 connected to a hand-held piezoelectric surgical tool comprising a tool handle 32 and a surgical tool tip 10 connected to the handle 32. The surgical tool tip 10 is typically detachably connected to the handle 32 and may be readily changed to suit various uses by the dental surgeon. The surgery unit 30 is typically configured to generate predetermine ultrasound frequencies, for example in the range of between 25-35 KHz suitable for operating safely on bone tissues, and the tool handle 32 is typically configured for generating micro-vibrations to drive the surgical tool tip 10 at the predetermined frequencies.

The surgical tool tip 10 is applied during use by a dental surgeon to cut into an alveolar bone 34 as shown in FIG. 2 of a patient. The present invention relates to the use of novel surgical tool tips 10 for the creation of a generally horizontal rectangular dental osteotomy site 36 as indicated in the boxed area of FIG. 2 in preparation for receiving a generally horizontal dental implant as described for example in PCT application no. PCT/AU2016/050339, the entire contents of which are hereby incorporated into this specification by reference. It has been found that horizontally-oriented dental implants provide significant technical performance advantages in areas such as improved osseointegration, longevity, reliability and maximum compressive force ranges under operating conditions over conventional short vertically-oriented implants.

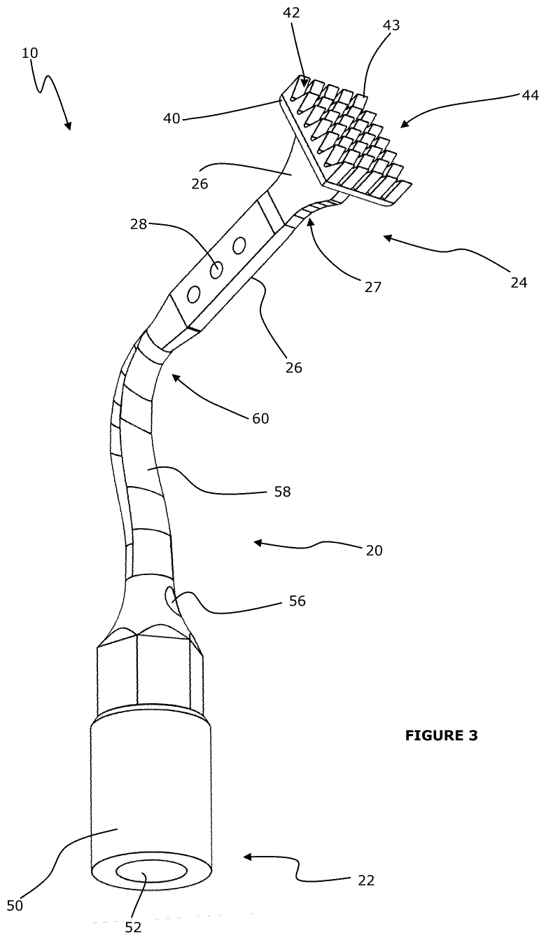

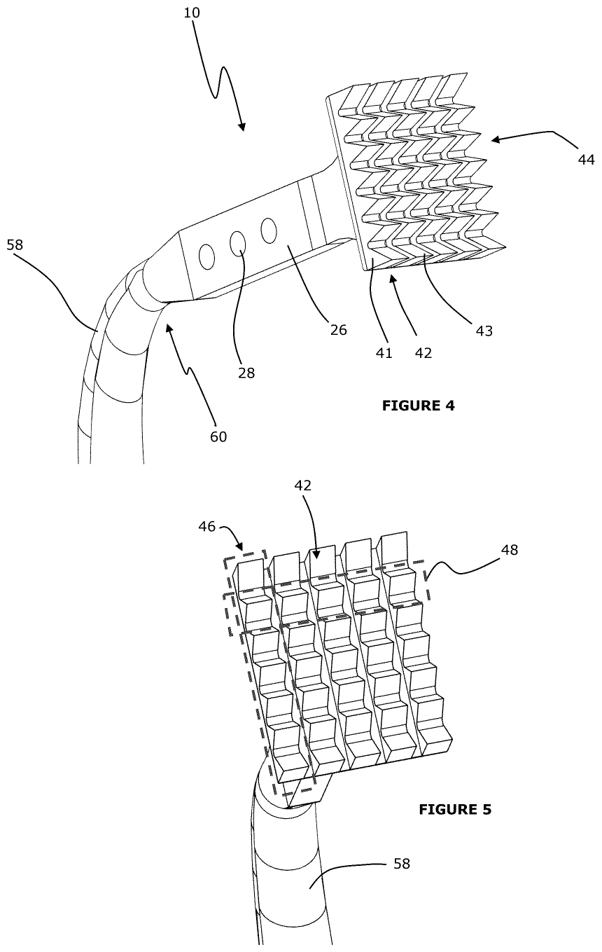

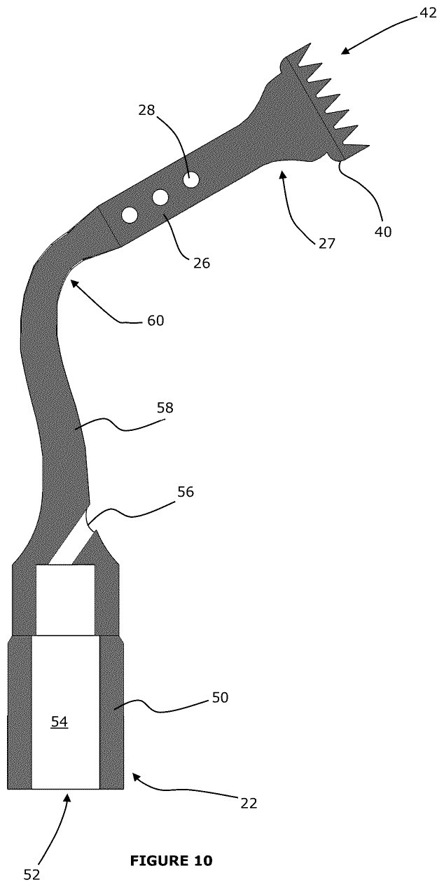

FIGS. 3 to 10 show a piezoelectric surgical tool tip 10 according to a preferred embodiment of the present invention. The tool tip 10 comprises a tool tip body 20 having two end portions; a proximal end portion 22 configured for connection with the tool handle 32 of a powered piezoelectric surgery unit 30 as described above and a distal end portion 24 configured for cutting into an alveolar bone or other suitable bone tissues of a patient by applying piezoelectric vibrations generated by the surgery unit 30 and handle 32.

In the preferred embodiment, the tool tip 10 is provided at the proximal end portion 22 with a generally cylindrical base 50 for connecting with a tool handle 32 and an opening 52 at the end of the base 50 for receiving water flow from the surgery unit 30 so as to provide irrigation to the surgical site 36 during use. The tool tip body 20 comprises an internal passageway 54 for receiving water flowing through the opening 52 and for guiding said water flow generally towards the distal end portion 24 of the tool tip body 20. In one configuration, the internal passageway 54 terminates at an outlet 56 along the tip body 20 and water continues to flow outwards following the contour of the tip body portion 58 towards the distal end portion 24. The tool tip body 20 comprises an elbow portion 60 which provides a generally 45 degree bend to assist with the application of the tool tip during use. The distal end portion 24 of the tool tip 10 comprises an outwardly extending arm 26 which extends from the elbow portion 60 and a tip end of the arm 26 locates a tool for operating on bone tissues during use. In the preferred embodiment, the tip end of the arm 26 is provided with a base 40 which is mounted substantially perpendicular to the arm 26, and a plurality of cutting projections 42 extend outwardly from the base 40. In one configuration, the plurality of cutting projections 42 can be said to be arranged in the form of an array 44, preferably the array 44 of cutting projections 42 is substantially rectangular and the cutting projections 42 are arranged linearly in orderly series of rows and columns. The orientation of the series of rows and columns can be defined relative to the orientation of the tool tip 10 when the tool tip body 20 is in a generally upright position.

In one configuration, the arm 26 is in the form of a flattened beam angled away from the elbow portion 60. The flattened beam has a general, non-limiting, thickness of about 0.75 mm in some embodiments, and the beam expands in width at a neck portion 27 to an enlarged head with increased width for greater surface area in connecting with the base 40. The arm 26 is also provided with one or more marking(s) located along the arm 26 to indicate a relative distance from the marking to the array 44 of cutting projections 42—this assists the dental surgeon to approximate a relative depth of the created osteotomy site during use. The embodiment of tool tip 10 shown in the Figures is provided with three such markings 28 in the form of cavities that extend right through the depth of the arm 26. This allows fluid flow through the markings 28 during use. It is to be understood that other marking types and number not described herein may be suitable without departing from the invention.

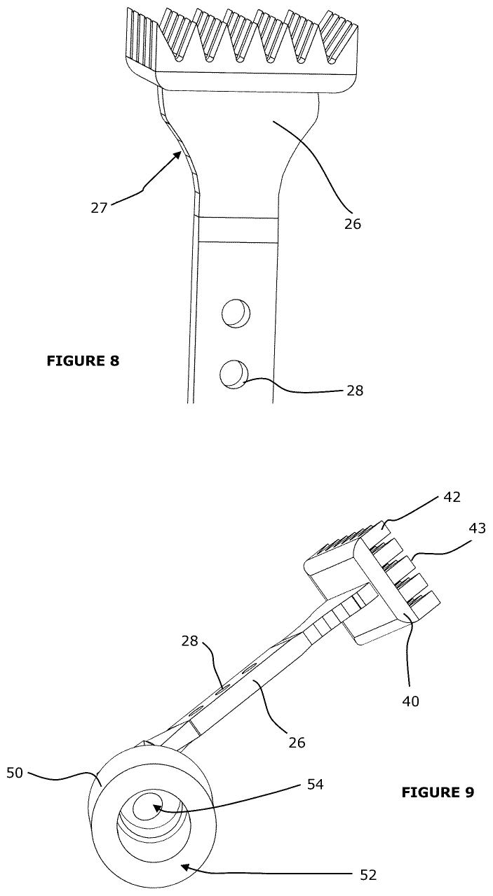

In the preferred embodiment, each cutting projections 42 is generally shaped in the form of a triangular prism. A lower portion 41 of each prism is seated on the base 40 and an upper vertex of the prism defines a cutting edge 43 for the tool tip 10. In the preferred embodiment, the array 44 of cutting projections 42 comprises a plurality of identical or similarly configured triangular prisms organised in a series of rows and columns. It is to be understood however that cutting projections 42 may be of any other suitable shapes and the array may also comprise cutting projections 42 having multiple different shapes all without departing from the spirit of the invention. The lower portion 41 of multiple adjacent cutting projections 42 may also be integrally formed or connected as seen in FIG. 7.

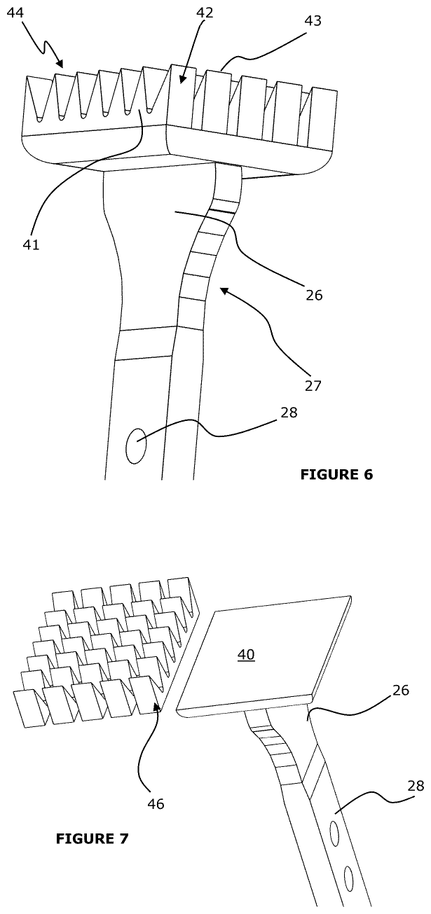

Turning to FIG. 5, the array 44 of similar or like cutting projections 42 can be said to be arranged in linear series of rows 48 and columns 46. The array 44 itself is of generally rectangular shape, but its orientation relative to the tool tip 10 when the tool tip body 20 is in a generally upright position can be distinguished between a “tall rectangular shape” and a “wide rectangular shape”. An example of an array 44 having a tall rectangular shape is shown in the embodiment seen in FIGS. 3 to 10, in which the length of the array 44 is greater than the width of the array 44. This can be achieved by having an array 44 in which the total number of rows 48 of cutting projections 42 is greater than the total number of columns 46 of cutting projections 42. In the preferred embodiment, the tall rectangular shaped array 44 has a width of about 4 mm and a length of about 5 mm. It is to be understood that these dimensions are provided merely for an exemplary embodiment, and are not intended to be limiting.

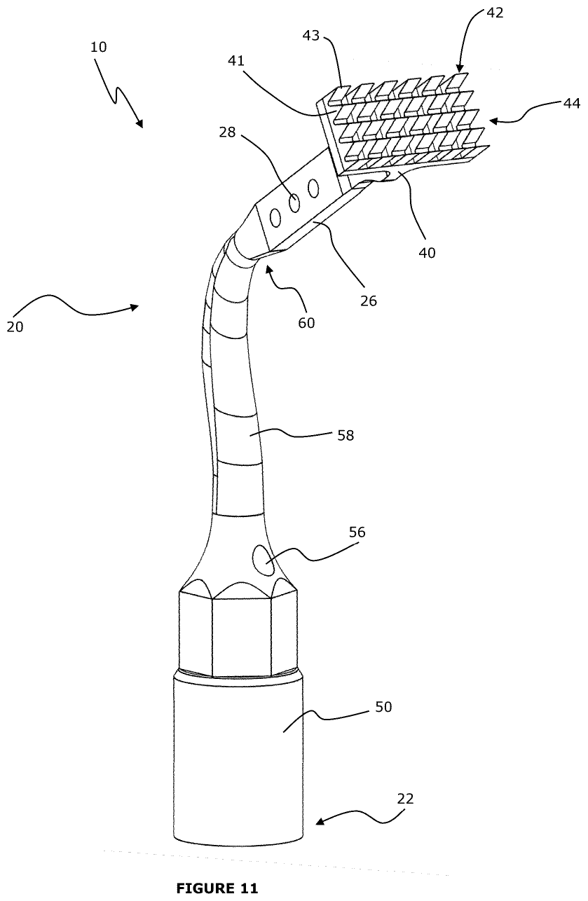

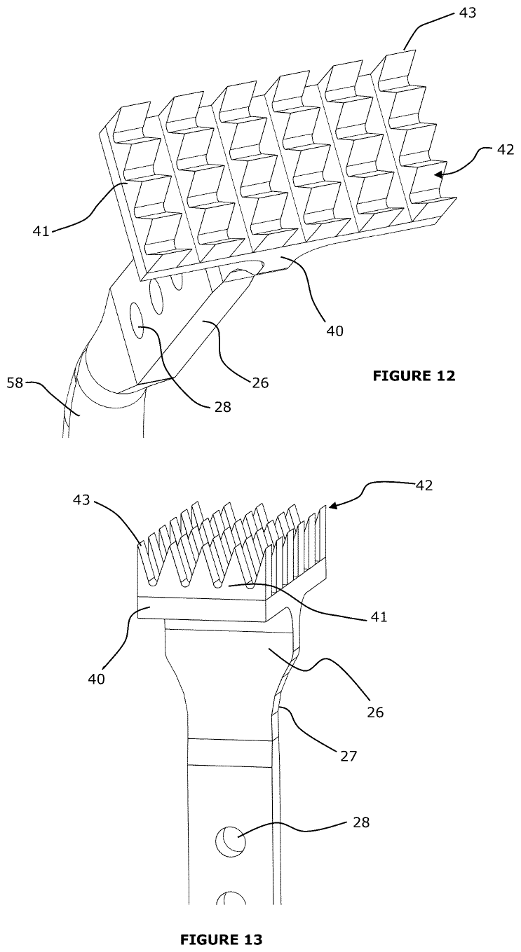

An example of an array 44 having a wide rectangular shape is shown in the embodiment as seen in FIGS. 11 to 13, in which the width of the array 44 is greater than the length of the array 44. This can be achieved by having an array 44 in which the total number of columns 48 of cutting projections 42 is greater than the total number of rows 46 of cutting projections 42. In the embodiment described, the wide rectangular shaped array 44 has a width of about 6 mm and a length of about 3 mm.

Examples of possible dimensions of the array 44 of cutting projections 42 for tall or wide rectangular shaped arrays 44 include a generally maximum width of about 6 mm and a generally maximum length of about 5 mm. In the preferred embodiment, each cutting projection 42 in the array 44 is configured with a height of about 1.2 mm. Again, it is to be understood that these dimensions are provided merely for an exemplary embodiment, and are not intended to be limiting.

The triangular prism cutting projections 42 of the tool tip 10 may be evenly spaced or distributed in the array as seen in the described embodiments. In some configurations, a gap of about 0.75 mm is provided between each adjacent peaks or cutting edges 43 of the triangular prisms in a given column. In relation to the use of triangular prisms as designated shapes for the cutting projections 42, prisms that fall on the edge sections of a row or column may be configured with only half of the triangular width yet maintain the same peak vertex height so as to provide an array with an even height across all the prisms. Again, any dimensional values provided herein are capable of variation without departing from the invention.

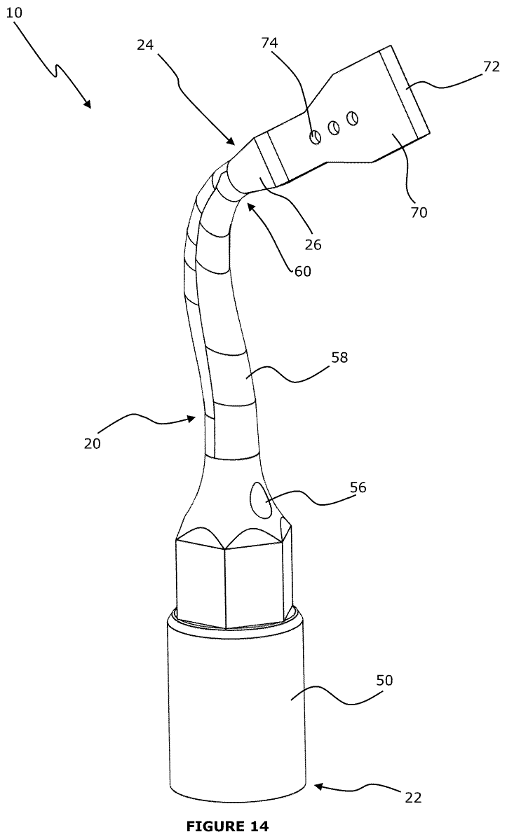

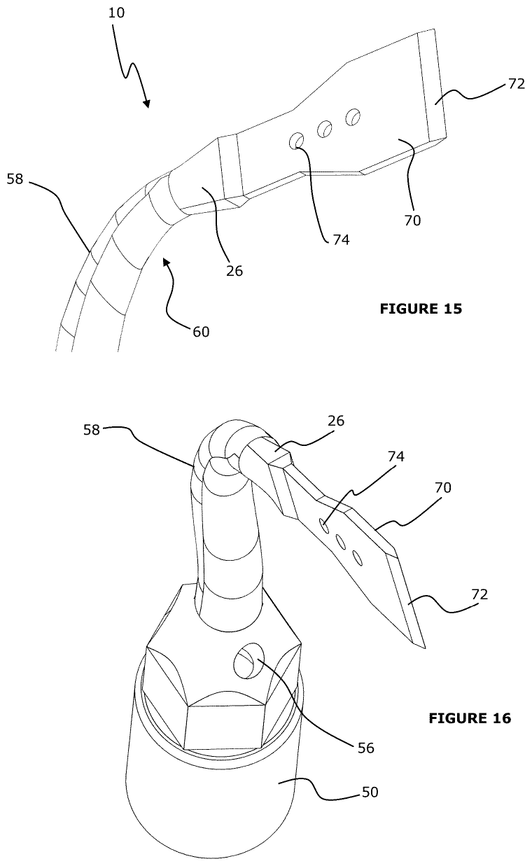

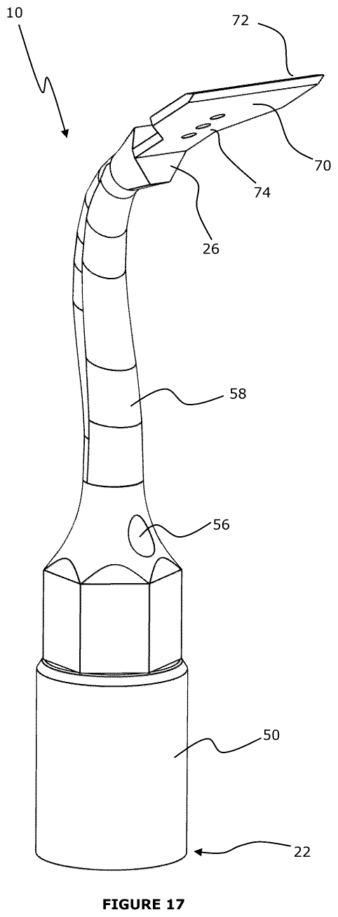

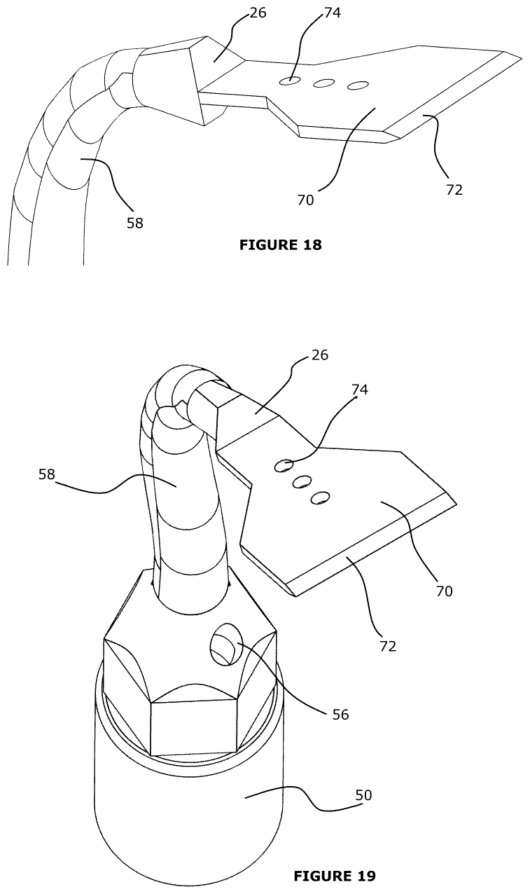

FIGS. 14 to 16 show an alternative embodiment of a tool tip body 20 having a chisel end portion extending from the arm 26 instead of an array 44 of cutting projections 42. The chisel end portion comprises a chisel blade 70 and a blade tip end 72 for cutting into the osteotomy site. In one embodiment, the chisel end portion is provided with markings 74 to indicate a relative distance from each marking to the blade tip end 72. The chisel end portion is configured such that the chisel blade 70 is oriented substantially vertically relative to the tool tip 10 when the tool tip body 20 is in an upright position. In an alternative embodiment as seen in FIGS. 17 to 19, the chisel end position can be configured such that the chisel blade 70 is oriented substantially horizontal relative to the tool tip 10 when the tool tip body 20 is in an upright position. Both types of chisel blade configurations described herein assists the dental surgeon in smoothing out any irregular surfaces in the osteotomy site created by the tool tips 10 using the array 44 of cutting projections 42 as described earlier.

It is to be appreciated that the tool tip body 20 as described herein can be integrally formed, such as 3D printed for example, or assembled from separate components. The whole or part of the tool tip body 20, including the array 44 of cutting projections 42, may be treated with a titanium nitride coating for improved wear resistance.

During use, an appropriate embodiment of the tool tip 10 of the present invention can be selected by a dental surgeon and detachably connected to the tool handle 32 of the piezoelectric surgery unit 30. A conduit supplying water and power from the piezoelectric surgery unit 30 is connected between the surgery unit 30 and the tool handle 32. When powered and connected, the surgery unit 30 is capable of generating appropriate ultrasound frequencies which is produced in the form of micro vibrations by piezoelectric mechanisms inside the tool handle 32, which in turn drives the connected tool tip 10 to effect a cutting action against the bone tissue 34. A continuous flow of water is provided through the conduit to the tool handle 32 and the tool tip 10, towards the distal end portion 24 or the chisel end portion of the tool tip body 20.

It is to be understood that the surgical tool tips 10 as described herein may be compatible with, and used with, any conventional piezoelectric dental surgery systems 30.

A method for creating a generally horizontally rectangular dental osteotomy site 36 in an alveolar bone 34 using the piezoelectric dental surgical surgery system and various surgical tool tips 10 embodiments as described above are also provided herein. The method of using the described tool tips 10 to create a generally horizontal rectangular osteotomy site 36 comprises the steps of:
providing a first piezoelectric surgical tool tip 10 in which the array 44 of cutting projections 42 is configured in the form of a tall rectangular shape, as described above, for example having a width of about 4 mm and a length of about 5 mm;
providing a second piezoelectric surgical tool tip 10 in which the array 44 of cutting projections 42 is configured in the form of a wide rectangular shape, as described above, for example having a width of about 6 mm and a length of about 3 mm;
applying piezoelectric vibrations generated from the surgery system to cut the alveolar bone 34 using the first piezoelectric surgical tool tip 10 in a first direction, which is substantially along a mesial-distal direction of the alveolar bone 34;
applying piezoelectric vibrations generated from the surgery system to cut the alveolar bone 34 using the second piezoelectric surgical tool tip 10 in a second direction, which is substantially perpendicular to the first direction; and
alternating applications of piezoelectric vibrations using the first and second piezoelectric surgical tool tips 10 to the alveolar bone 34 until a generally horizontally rectangular osteotomy site 36 with a desired depth is achieved. In some cases, the desired depth is about 5.5 mm.

When the tool tip 10 is used to create the rectangular dental osteotomy site 36 per the method described above, it is to be appreciated that the base 40, from which the cutting projections 42 emanate, also acts as a platform to limit the depth of the osteotomy site 36 being cut so as to guard against causing inadvertent damage to vital anatomical structures of the alveolar bone 34 during surgery.

It is to be understood that the relative order of the method steps described and the relative direction of travel of the tip tools 10 are capable of variation without departing from the spirit of the invention.

Additional steps to complete the osteotomy site could include the use of a piezoelectric surgical tool tip 10 with a chisel tip end as described in relation to FIGS. 14 to 19 to smooth out any irregular surfaces within the site.

The method described herein provides an effective way of creating a rectangular osteotomy site to accommodate the specialised horizontally-oriented dental implants in a safe and consistent manner. The use of two alternating tool tips 10, each with its own set of tall or wide orientation, is a key method to the effectiveness of this method.

In the description and drawings of this embodiment, same reference numerals are used as have been used in respect of the first embodiment, to denote and refer to corresponding features.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A piezoelectric surgical tool tip, comprising:
a tool tip body having a proximal end configured for connection with a tool handle of a powered piezoelectric surgery unit and a distal end configured for cutting into an alveolar bone of a patient using micro-vibrations generated by the surgery unit and tool handle,
the distal end comprising an outwardly extending arm and a base on which extends a plurality of cutting projections arranged in the form of an array, wherein the base is mounted substantially perpendicular to the arm and the array is substantially rectangular in shape with a maximum width of about 6 mm and a maximum length of about 5 mm.

2. The surgical tool tip according to claim 1, wherein the cutting projections of the array are arranged linearly in series of rows and columns, defined relative to the orientation of the tool tip when the tool tip body is in an upright position.

3. The surgical tool tip according to claim 2, wherein the array of cutting projections is configured in the form of a tall rectangular shape in which the number of rows is greater than the number of columns, and wherein the width of the array is about 4 mm and the length of the array is about 5 mm.

4. The surgical tool tip according to claim 3, wherein the number of columns of cutting projections is five and the number of rows of cutting projections is seven.

5. The surgical tool tip according to claim 2, wherein the array of cutting projections is configured in the form of a wide rectangular shape in which the number of columns is greater than the number of rows, and wherein the width of the array is about 6 mm and the length of the array is about 3 mm.

6. The surgical tool tip according to claim 5, wherein the number of columns of cutting projections is six and the number of rows of cutting projections is five.

7. The surgical tool tip according to claim 1, wherein each of the plurality of cutting projections is generally shaped in the form of a triangular prism and oriented such that an upper vertex of the prism defines a cutting edge.

8. The surgical tool tip according to claim 7, wherein a row or column of like triangular prism cutting projections is formed such that their triangular bases are integrally connected.

9. The surgical tool tip according to claim 7, wherein a gap of about 0.75 mm is provided between the cutting edges of adjacent like triangular prisms in the array.

10. The surgical tool tip according to claim 1, wherein each cutting projection is configured with a height of about 1.2 mm.

11. The surgical tool tip according to claim 1, wherein the arm is in the form of a flattened beam, and wherein the arm is provided with one or more marking(s) located along the beam to indicate a relative distance from the marking to the array of cutting projections.

12. The surgical tool tip according to claim 11, wherein the or each marking is a cavity that extends through the arm.

13. The surgical tool tip according to claim 11, wherein the arm is configured with a thickness of about 0.75 mm.

14. The surgical tool tip according to claim 1, wherein the tool tip body further comprises an internal passageway for receiving water flow at the proximal end and guiding said water flow generally towards the distal end.

15. The surgical tool tip according to claim 1, preceding claims, wherein the array of cutting projections and the base are integrally formed.

16. The surgical tool tip according to claim 1, wherein the tool tip body is treated with a titanium nitride coating.

17. The surgical tool tip according to claim 1, wherein the tool tip body is configured to be detachably connectable with the tool handle of the powered piezoelectric surgery unit.

18. A dental surgical system, comprising:
a piezoelectric surgery unit configured for generating predetermined ultrasound frequencies;
a tool handle connectable to the piezoelectric surgery unit to generate micro-vibrations at the predetermined frequencies; and
a piezoelectric surgical tool tip according to claim 1 configured for connection with the tool handle for cutting into an alveolar bone of a patient using the generated micro-vibrations.

19. A method of creating a generally horizontally rectangular dental osteotomy site in an alveolar bone, the method comprising the steps of:
providing a first piezoelectric surgical tool tip according to claim 1 in which the array of cutting projections is configured in the form of a tall rectangular shape having a width of about 4 mm and a length of about 5 mm;
providing a second piezoelectric surgical tool tip according to claim 1 in which the array of cutting projections is configured in the form of a wide rectangular shape having a width of about 6 mm and a length of about 3 mm;
applying piezoelectric vibrations to cut the alveolar bone using the first piezoelectric surgical tool tip in a first direction, which is substantially along a mesial-distal direction of the alveolar bone;
applying piezoelectric vibrations to cut the alveolar bone using the second piezoelectric surgical tool tip in a second direction, which is substantially perpendicular to the first direction; and
alternating applications of piezoelectric vibrations using the first and second piezoelectric surgical tool tips to the alveolar bone until a generally horizontally rectangular osteotomy site with a desired depth is achieved.

20. The method of creating a rectangular dental osteotomy site according to claim 19, further comprising the step of:
applying piezoelectric vibrations to the horizontally rectangular osteotomy site using a piezoelectric surgical tool tip with a chisel tip end to smooth out any irregular surfaces within the site.
